(19) United States
(12) Patent Application Publication
(10) Pub. No.: US 2021/0393391 A1
(43) Pub. Date: Dec. 23, 2021

(54) OSSEOINTEGRATING RING FOR COUPLING OF BONE CONDUCTION DEVICE

(71) Applicant: Cochlear Limited, Macquarie University, NSW (AU)

(72) Inventor: Wim Bervoets, Macquarie University (AU)

(21) Appl. No.: 17/293,013

(22) PCT Filed: Apr. 27, 2020
(86) PCT No.: PCT/IB2020/053955

§ 371 Date: May 11, 2021

Related U.S. Application Data
(60) Provisional application No. 62/842,137, filed on May 2, 2019.

(51) Int. Cl. A61F 2/00; H04R 25/00

(52) U.S. Cl. CPC A61F 2/0077; H04R 25/606; A61F 2002/183; A61F 2002/0086; H04R 2460/13

(57) ABSTRACT

An apparatus is provided which includes a planar body including an osseointegrating material and at least one hole configured to receive at least one protrusion of a subcutaneous acoustic transducer device. The body is configured to be implanted in contact with a portion of a bone of a recipient.

### BACKGROUND

### Field

The present application relates generally to implantable auditory prostheses, and more specifically systems and methods utilizing an osseointegrating element for mechanically coupling the acoustic prosthesis to the skull of the recipient.

### Description of the Related Art

Hearing loss, which may be due to many different causes, is generally of two types, conductive and/or sensorineural. Conductive hearing loss occurs when the normal mechanical pathways of the outer and/or middle ear are impeded, for example, by damage to the ossicular chain or ear canal. Sensorineural hearing loss occurs when there is damage to the inner ear, or to the nerve pathways from the inner ear to the brain. Auditory prostheses of various types are widely used to improve the lives of users. Such devices include, for example, hearing aids, cochlear implants, bone conduction implants, middle ear implants, and electro-acoustic devices.

Individuals who suffer from conductive hearing loss typically have some form of residual hearing because the hair cells in the cochlea are undamaged. As a result, individuals suffering from conductive hearing loss might receive an auditory prosthesis that generates mechanical motion of the cochlea fluid instead of a hearing aid, based on the type of conductive loss, amount of hearing loss and customer preference. An example of such prostheses includes bone conduction devices which convert a received sound into vibrations. The vibrations are transferred through teeth and/or bone to the cochlea, causing generation of nerve impulses, which result in the perception of the received sound. Bone conduction devices can be coupled using a direct percutaneous implant and abutment, or using transcutaneous solutions, which can contain an active or passive implant component, or other mechanisms to transmit sound vibrations through the skull bones, such as through vibrating the ear canal walls or the teeth.

Forms of these auditory prostheses which are “mostly implantable,” “fully implantable,” or “totally implantable” have most or all the components of the auditory prosthesis configured to be implanted under the skin/tissue of the recipient and the auditory prosthesis operates, for at least a finite period of time, without the need of an external device. An external device can be used to charge the internal battery, to supplement the performance of the implanted microphone/system, or for when the internal battery no longer functions. Such devices have the advantage of allowing the user to have a superior aesthetic result, as the recipient is visually indistinguishable in day-to-day activities from individuals that have not received such devices. Such devices also have a further advantage in generally being inherently waterproof, allowing the recipient to shower, swim, and so forth without needing to take any special measures.

While conventional auditory prostheses use externally disposed microphone assemblies, certain mostly, fully, or totally implantable auditory prostheses use subcutaneously implantable microphone assemblies. Such microphone assemblies are configured to be positioned (e.g., in a surgical procedure) beneath the skin and on, within, or proximate to the recipient's skull and at a location that facilitates the receipt of acoustic signals by the microphone assembly once implanted (e.g., at a location between the recipient's skin and skull, rearward and upward of the recipient's ear or in the mastoid region).

### SUMMARY

In one aspect disclosed herein, an apparatus is provided which comprises a planar body comprising an osseointegrating material and at least one hole configured to receive at least one protrusion of a subcutaneous acoustic transducer device. The body is configured to be implanted in contact with a portion of a bone of a recipient.

In another aspect disclosed herein, a method is provided which comprises generating acoustic vibrations in response to ambient sound from an environment of a recipient. The method further comprises transmitting the acoustic vibrations to a planar interface in mechanical communication with a bone of the recipient. The planar interface comprises a surface receiving the acoustic vibrations. The method further comprises transmitting the acoustic vibrations from the planar interface to the bone of the recipient.

In still another aspect disclosed herein, an apparatus is provided which comprises a plurality of cutting edges configured to rotated about an axis to machine a portion of a bone of a recipient. The plurality of cutting edges comprises at least a first set of the cutting edges configured to machine a first planar surface on the bone. The first planar surface is recessed relative to a surrounding region of the bone.

### BRIEF DESCRIPTION OF THE DRAWINGS

Embodiments are described herein in conjunction with the accompanying drawings, in which:

FIG. 1A schematically illustrates a portion of an example transcutaneous bone conduction auditory prosthesis implanted in a recipient in accordance with certain embodiments described herein;

FIG. 1B schematically illustrates a portion of another example transcutaneous bone conduction auditory prosthesis implanted in a recipient in accordance with certain embodiments described herein;

FIG. 2A schematically illustrates a top view of an example apparatus in accordance with certain embodiments described herein.

FIG. 2B schematically illustrates a perspective view of the example apparatus of FIG. 2A;

FIG. 2C schematically illustrates a perspective view of the example apparatus of FIG. 2A positioned within a recess of the bone in accordance with certain embodiments described herein;

FIGS. 3A-3L schematically illustrate various example apparatus in accordance with certain embodiments described herein;

FIGS. 4A-4C schematically illustrate an example implanted apparatus and an example subcutaneous acoustic transducer device in accordance with certain embodiments described herein;

FIG. 5 schematically illustrates an example recess within the bone in accordance with certain embodiments described herein;

FIG. 6 schematically illustrates an example drilling apparatus configured to be used during implantation of the example osseointegrating apparatus in accordance with certain embodiments described herein; and

FIG. 7 is a flow diagram of an example method in accordance with certain embodiments described herein.

### DETAILED DESCRIPTION

Certain embodiments described herein provide an osseointegrating element (e.g., ring) to facilitate the coupling of a bone conduction device to a recipient's skull. The osseointegrating element of certain embodiments comprises a planar body and at least one hole configured to receive (e.g., to be in mechanical communication with) at least one protrusion of the bone conduction device. The osseointegrating element of certain embodiments comprises a low-profile interface to the cortical bone of the recipient's skull with an open-bottom design that reduces the possibility of infection risk and that utilizes an inner metal surface of the osseointegrating element to contact an outer metal surface of the bone conduction device (e.g., a metal-to-metal contour contact between the osseointegrating element and the bone conduction device).

The osseointegrating element of certain embodiments described herein provides a larger anchoring region with the recipient's bone as compared to single-point fixation, thereby advantageously providing less sensitivity to trauma and loosening fixation torques. In addition, the osseointegrating element of certain embodiments is configured to be affixed to the recipient's skull with a small depth penetration (e.g., not extending below the upper cortical layer), which is advantageously less invasive and/or advantageously compatible with use in various contexts (e.g., pediatrics). In certain embodiments, the interface between the osseointegrating element and the recipient's skull is wholly or predominantly within the cortical region of the recipient's skull, thereby enhancing (e.g., maximizing) sound conduction efficiency between the osseointegrating element and the recipient's skull. In certain embodiments, the osseointegrating element advantageously provides an interface between the bone conduction device and the recipient's skull that is less dependent (e.g., not dependent; minimally dependent) on the quality of the surgical implantation technique, that can facilitate more consistent and reproducible device performance, and/or reduces the risk of altering the device-to-bone interface and vibration transfer (e.g., device performance) upon re-surgery (e.g., during a procedure in which the active bone conduction device is replaced while the osseointegrating element remains in place) or application of external loads. In certain embodiments, implantation of the osseointegrating element is advantageously simpler (e.g., less complicated; comprises fewer surgical steps) than for other bone conduction systems utilizing a bone screw fixture.

The teachings detailed herein are applicable, in at least some embodiments, to any type of auditory prosthesis utilizing a subcutaneous acoustic implant (e.g., microphone; actuator assembly), the auditory prosthesis including but not limited to: electro-acoustic electrical/acoustic systems, cochlear implant devices, implantable hearing aid devices, middle ear implant devices, bone conduction devices (e.g., active bone conduction devices; passive bone conduction devices, percutaneous bone conduction devices; transcutaneous bone conduction devices), Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components. Embodiments can include any type of auditory prosthesis that can utilize the teachings detailed herein and/or variations thereof. In some embodiments, the teachings detailed herein and/or variations thereof can be utilized in other types of prostheses beyond auditory prostheses.

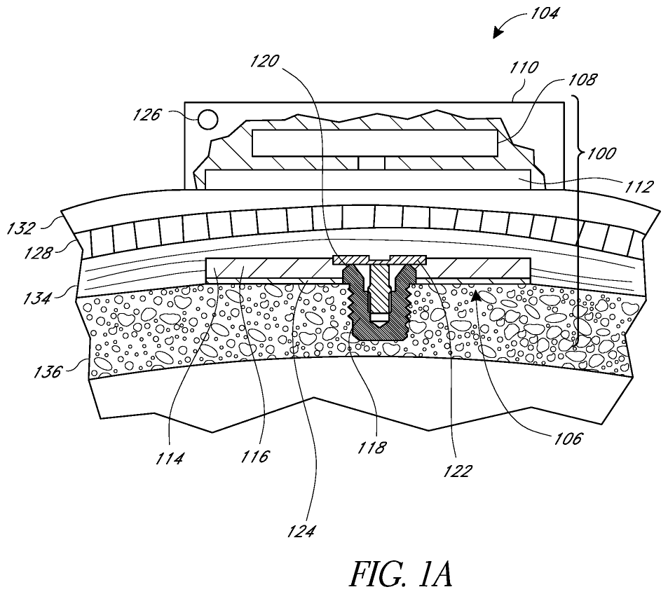

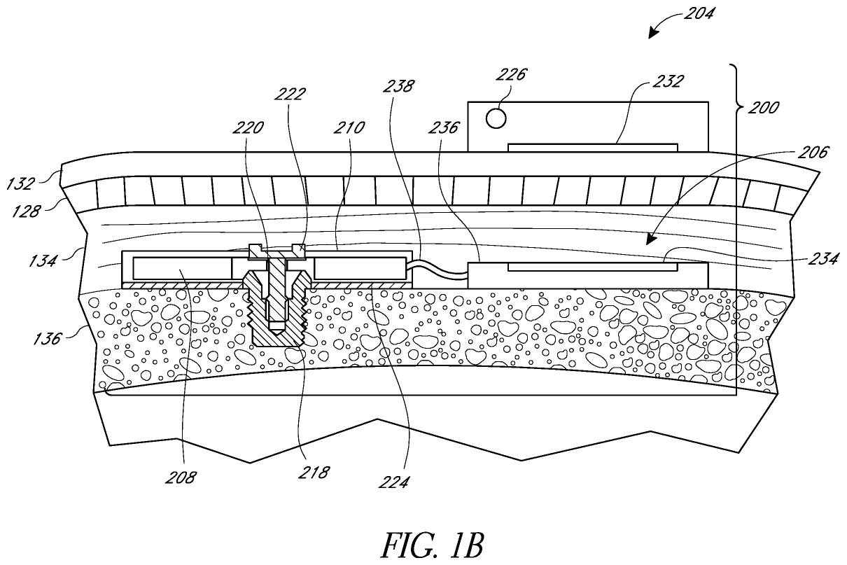

FIG. 1A schematically illustrates a portion of an example transcutaneous bone conduction auditory prosthesis 100 implanted in a recipient in accordance with certain embodiments described herein. FIG. 1B schematically illustrates a portion of another example transcutaneous bone conduction auditory prosthesis 200 implanted in a recipient in accordance with certain embodiments described herein.

The example transcutaneous bone conduction auditory prosthesis 100 of FIG. 1A includes an external portion 104 and an implantable portion 106. The transcutaneous bone conduction device 100 of FIG. 1A is a passive transcutaneous bone conduction device in that a vibrating actuator 108 is located in the external portion 104 and delivers vibrational stimuli through the skin 132 to the skull 136. The vibrating actuator 108 is located in the housing 110 of the external portion 104, and is coupled to a plate 112. The plate 112 of certain embodiments comprises a permanent magnet and/or is configured to generate and/or to be reactive to a magnetic field, or otherwise to permit the establishment of a magnetic attraction between the external portion 104 and the implantable portion 106 sufficient to hold the external portion 104 against the skin 132 of the recipient.

For example, the vibrating actuator 108 can comprise a device that converts electrical signals into vibration. In operation, a sound input element 126 (e.g., external microphone) converts sound into electrical signals. Specifically, the transcutaneous bone conduction device 100 provides these electrical signals to the vibrating actuator 108, via a sound processor (not shown) that processes the electrical signals, and then provides those processed signals to the vibrating actuator 108. The vibrating actuator 108 converts the electrical signals into vibrations. Because the vibrating actuator 108 is mechanically coupled to the plate 112, the vibrations are transferred from the vibrating actuator 108 to the plate 112. The implantable plate assembly 114 is part of the implantable portion 106, and can be made of a ferromagnetic material (e.g., a permanent magnet) that is configured to generate and/or to be reactive to a magnetic field, or otherwise to permit the establishment of a magnetic attraction between the external portion 104 and the implantable portion 106 sufficient to hold the external portion 104 against the skin 132 of the recipient. Accordingly, vibrations produced by the vibrating actuator 108 of the external portion 104 are transferred from the plate 112 across the skin 132 to the implantable plate 116 of the implantable plate assembly 114. This can be accomplished as a result of mechanical conduction of the vibrations through the skin 132, resulting from the external portion 104 being in direct contact with the skin 132 and/or from the magnetic field between the two plates 112, 116. These vibrations are transferred without a component penetrating the skin 132, fat 128, or muscular 134 layers on the head.

As can be seen in FIG. 1A, the implantable plate assembly 114 is substantially rigidly attached to a bone fixture 118 in this example. The implantable plate assembly 114 includes a through hole 120 that is contoured to the outer contours of the bone fixture 118, e.g., a bone fixture 118 that is secured to the bone 136 of the skull. This through hole 120 thus forms a bone fixture interface section that is contoured to the exposed section of the bone fixture 118. In an example, the through hole 120 and the bone fixture 118 are sized and dimensioned such that at least a slip fit or an interference fit exists with respect to the implantable plate assembly 114 and the bone fixture 118. The plate screw 122 is used to secure the implantable plate assembly 114 to the bone fixture 118. As can be seen in FIG. 1A, the head of the plate screw 122 is larger than the through hole 120 of the implantable plate assembly 114, and thus the plate screw 122 positively retains the implantable plate assembly 114 to the bone fixture 118. In certain examples, a silicon layer 124 is located between the implantable plate 116 and the bone 136 of the skull.

FIG. 1B schematically illustrates a portion of another example transcutaneous bone conduction auditory prosthesis 200 implanted in a recipient in accordance with certain embodiments described herein. The transcutaneous bone conduction auditory prosthesis 200 includes an external portion 204 and an implantable portion 206 that is implanted beneath the various tissue layers shown. For example, the external portion 204 corresponds to the external portion 104 detailed above, and the implantable portion 206 corresponds to the implantable portion 106 detailed above.

The transcutaneous bone conduction device 200 of FIG. 1B is an active transcutaneous bone conduction device in that the vibrating actuator 208 is located in the implantable portion 206. For example, a vibratory element in the form of a vibrating actuator 208 can be located in the housing 210 of the implantable portion 206. Much like the vibrating actuator 108 described above with respect to the example transcutaneous bone conduction device of FIG. 1A, the vibrating actuator 208 of FIG. 1B is configured to convert electrical signals into vibrations. The vibrating actuator 208 is in direct contact with the outer surface of the recipient's skull (e.g., the vibrating actuator 208 is in substantial contact with the recipient's bone 136 such that vibration forces from the vibrating actuator 208 are communicated from the vibrating actuator 208 to the recipient's bone 136). In certain embodiments, there may be one or more thin non-bone tissue layers (e.g., a silicon layer 224) between the vibrating actuator 208 and the recipient's bone 136 (e.g., bone tissue) while still permitting sufficient support so as to allow efficient communication of the vibration forces generated by the vibrating actuator 208 to the recipient's bone 136.

The external portion 204 includes a sound input element 226 (e.g., external microphone) that converts sound into electrical signals. Specifically, the transcutaneous bone conduction device 200 provides these electrical signals to the vibrating actuator 208, or to a sound processor (not shown) that processes the electrical signals, and then provides those processed signals to the implantable portion 206 through the skin 136 of the recipient via a magnetic inductance link. For example, a transmitter coil 232 of the external portion 204 can transmit inductance signals to an implanted receiver coil 234 located in a second housing 236 of the implantable portion 206. Components (not shown) in the second housing 236, such as, for example, a signal generator or an implanted sound processor, then generate electrical signals to be delivered to the vibrating actuator 208 via electrical lead assembly 238. The vibrating actuator 208 coverts the electrical signals into vibrations. In certain embodiments, the vibrating actuator 208 may be positioned with such proximity to the second housing 236 that the electrical leads 238 are not present (e.g., the first housing 210 and the second housing 238 are the same single housing containing the vibrating actuator 208, the receiver coil 234, and other components, such as, for example, a signal generator or a sound processor).

The vibrating actuator 208 is mechanically coupled to the housing 210. The housing 210 and the vibrating actuator 208 collectively form a vibrating element. The housing 210 is substantially rigidly attached to the bone fixture 218. In this regard, the housing 210 includes a through hole 220 that is contoured to the outer contours of the bone fixture 218. The housing screw 222 is used to secure the housing 210 to the bone fixture 218. As can be seen in FIG. 1B, the head of the plate screw 22 is larger than the through hole 220 of the housing 210, and thus the plate screw 222 positively retains the housing 210 to the bone fixture 218. In certain examples, a silicon layer 224 is located between the housing 210 and the bone 136 of the skull.

The example transcutaneous bone conduction auditory prosthesis 100 of FIG. 1A comprises an external sound input element 126 (e.g., external microphone) and the example transcutaneous bone conduction auditory prosthesis 200 of FIG. 1B comprises an external sound input element 226 (e.g., external microphone). Other example auditory prostheses (e.g., totally implantable transcutaneous bone conduction devices) in accordance with certain embodiments described herein can replace the external sound input element 126, 226 with a subcutaneously implantable sound input assembly (e.g., implanted microphone).

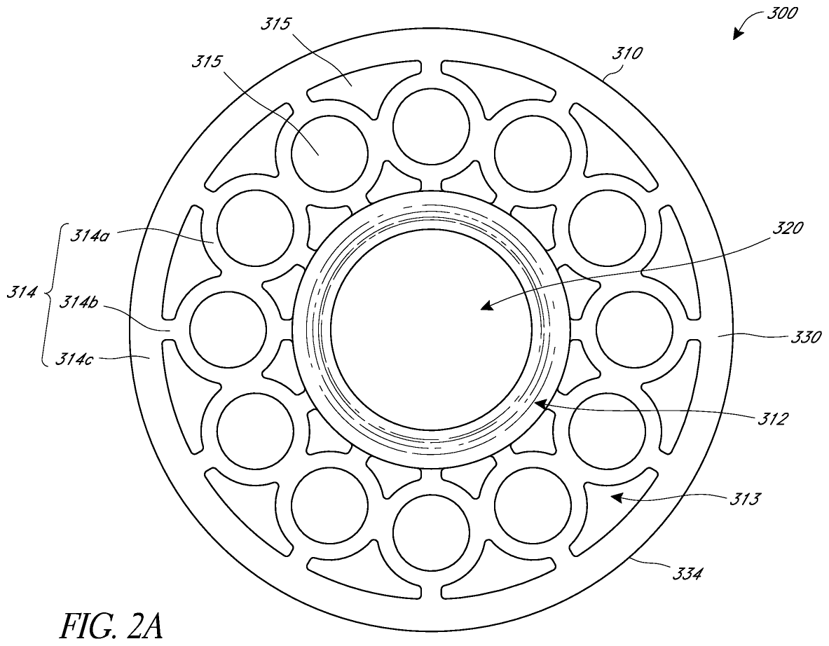

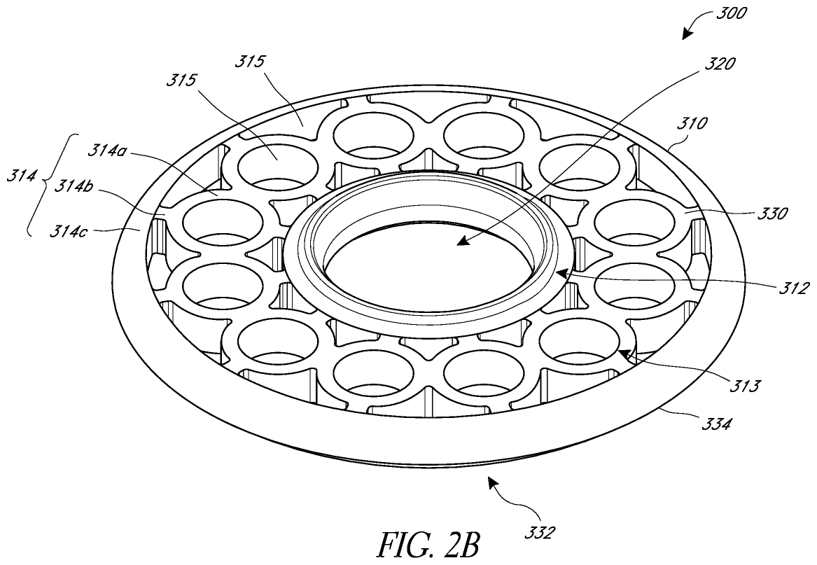

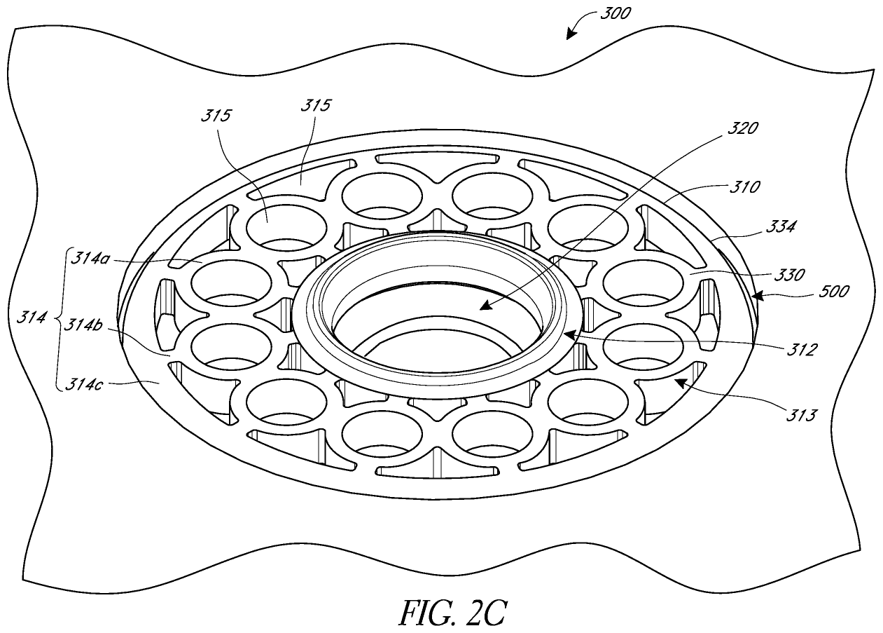

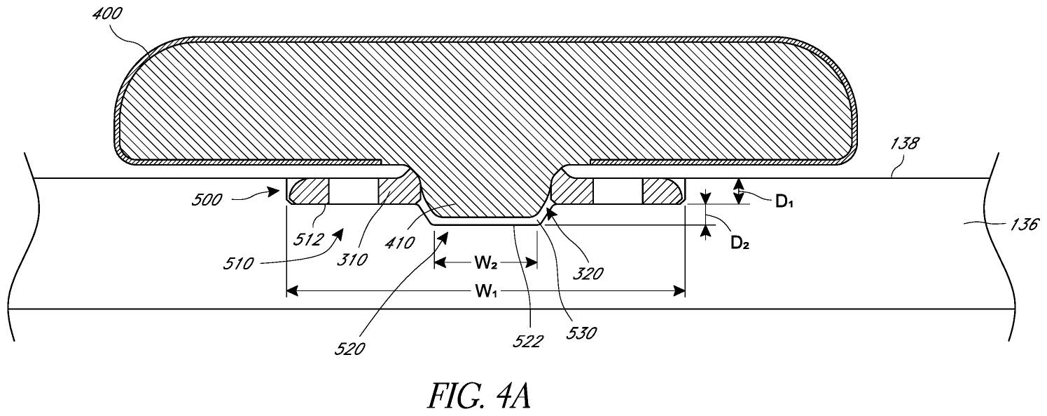

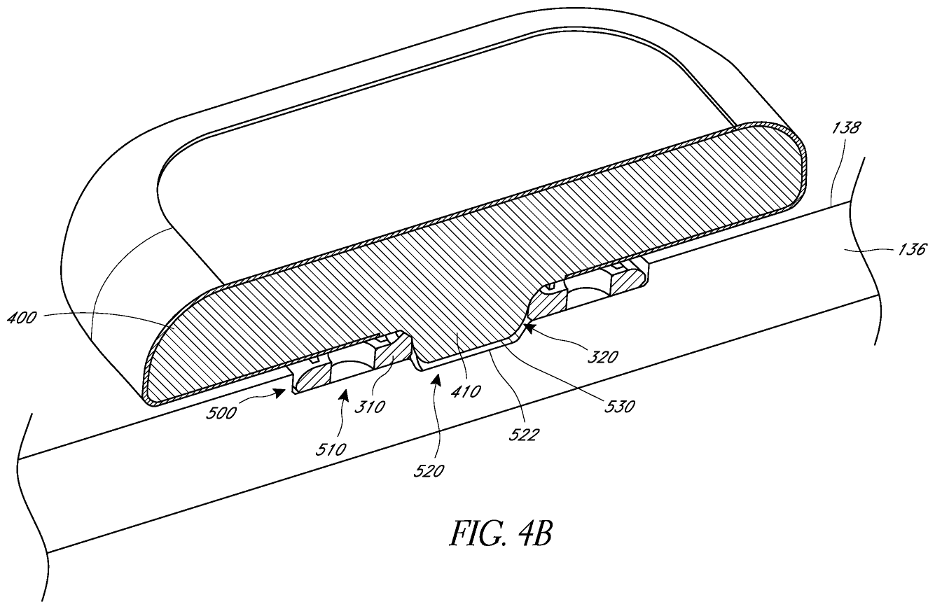

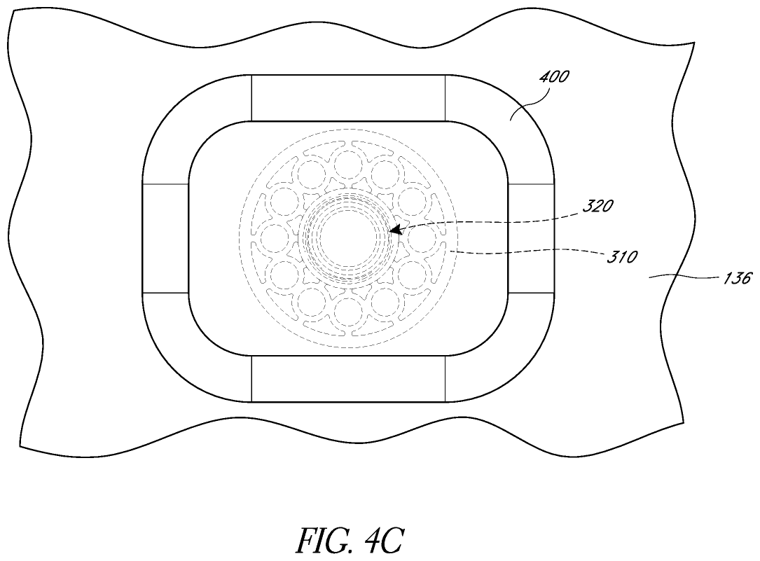

FIG. 2A schematically illustrates a top view of an example apparatus 300 in accordance with certain embodiments described herein. FIG. 2B schematically illustrates a perspective view of the example apparatus 300 of FIG. 2A. The example apparatus 300 comprises a planar body 310 comprising an osseointegrating material and at least one hole 320 configured to receive at least one protrusion 410 of a subcutaneous acoustic transducer device 400 (see, e.g., FIGS. 4A-4C) (e.g., the device 400 comprising an implantable plate assembly 114; comprising an implantable vibrating actuator 208). The body 310 is configured to be implanted in contact with a portion of a bone 136 of a recipient. FIG. 2C schematically illustrates a perspective view of the example apparatus 300 of FIG. 2A positioned within a recess 500 of the bone 136 in accordance with certain embodiments described herein.

In certain embodiments, the body 310 is configured to be between the acoustic transducer device 400 and the portion of the bone 136 (e.g., when the body 310 is implanted). For example, the acoustic transducer device 400 can comprise a vibrating actuator 208 and the body 310 can be configured to transmit acoustic vibrations from the vibrating actuator 208, through the at least one protrusion 410, to the portion of the bone 136. For another example, the acoustic transducer device 400 can comprise an implantable microphone and the body 310 can be configured to provide sufficient vibration transfer between the microphone and the bone 136 to facilitate noise cancellation to the microphone (e.g., from other electronics of the acoustic transducer device 400). By mechanically coupling the microphone to the portion of the bone 136, the body 310 of certain embodiments provides stability and sufficient mass to at least partially reduce a resonance frequency of the microphone and/or to at least partially tamp down a noise contribution to the acoustic signals received by the microphone.

In certain embodiments, the osseointegrating material is selected from a group consisting of: titanium, titanium alloy, tantalum, and tantalum alloys. As schematically illustrated by FIGS. 2A-2C, the body 310 of certain embodiments is circular and planar (e.g., disc-like; flat; extending along a body plane), while in certain other embodiments, the body 310 has other shapes (e.g., non-circular; parallelepiped; rectilinear; triangular; polygonal; slab-like) and/or is non-planar (e.g., curved). In certain embodiments, the body 310 has a shape that is symmetric about at least one plane (e.g., a plane perpendicular to the body plane), while in certain other embodiments, the body 310 has a shape that is asymmetric about at least one plane (e.g., a plane perpendicular to the body plane). For example, the body 310 can have an asymmetric shape such that the apparatus 300 fits at least partially within a corresponding asymmetric recess 400 in the bone 136. In certain embodiments, the body 310 has an outer width (e.g., outer diameter) in a range of 10 millimeters to 30 millimeters (e.g., in a range of 15 millimeters to 16 millimeters). In certain embodiments, the outer width of the body 310 is configured to provide efficient transfer of sounds (e.g., high-frequency sounds; low-frequency sounds; sounds within a predetermined range of frequencies) across the interface between the apparatus 300 and the recipient's bone 136.

As schematically illustrated by FIGS. 2A-2C, the second portion 313 of the body 310 of certain embodiments comprises a first surface 330 and a second surface 332. In certain embodiments, the first surface 330 is configured to face towards the acoustic transducer device 400 when the acoustic transducer device 400 and the apparatus 300 are mechanically coupled to one another, and the second surface 332 is configured to face towards and contact the portion of the bone 136 (e.g., when the body 310 is implanted). In certain embodiments, the first surface 330 and the second surface 332 are substantially parallel to one another, while in certain other embodiments, the first surface 330 and the second surface 332 are non-parallel to one another. The body 310 of certain embodiments has a thickness between the first surface 330 and the second surface 332, the thickness in a range of 1 millimeter to 3 millimeters (e.g., less than 1.5 millimeters).

As schematically illustrated by FIGS. 2A-2C, the at least one hole 320 of certain embodiments comprises a hole 320 extending from the first surface 330 to the second surface 332. The hole 320 of certain embodiments has a width (e.g., an inner diameter) in a range of 3 millimeters to 20 millimeters (e.g., 5 millimeters). The hole 320 of certain embodiments is circular (e.g., in a plane parallel to a body plane of the body 310), while in certain other embodiments, the hole 320 has other shapes (e.g., non-circular; rectilinear; triangular; polygonal) (e.g., in a plane parallel to a body plane of the body 310). In certain embodiments, the hole 320 is symmetric about at least one plane (e.g., a plane perpendicular to the body plane), while in certain other embodiments, the hole 320 is asymmetric about at least one plane (e.g., a plane perpendicular to the body plane). For example, the hole 320 can have a circular inner surface that is configured to be in mechanical communication with (e.g., mate with) a corresponding protrusion 410 of the acoustic transducer device 400. For another example, the hole 320 can have a non-circular shape that is configured to be in mechanical communication with (e.g., mate with) a corresponding protrusion 410 of the acoustic transducer device 400, so as to maintain a predetermined orientation of the acoustic transducer device 400 with the body 310.

In certain embodiments, the body 310 comprises a first portion 312 surrounding the at least one hole 320 and a second portion 313 surrounding the first portion 312. As schematically illustrated by FIGS. 2A-2C, in certain embodiments, the first portion 312 has a first density and the second portion 313 has a second density less than the first density. For example, the first density and the second density can be configured to facilitate transfer of acoustic vibrations from the at least one protrusion 410 to the bone 136. In certain embodiments, the first portion 312 has a first fraction of open regions and the second portion 313 has a second fraction of open regions greater than the first fraction, and the first fraction and the second fraction can be configured to facilitate osseointegration of the body 310 with the bone 136. For example, the first portion 312 can be solid (e.g., the first fraction of open regions is equal to zero). In certain embodiments, the second density of the second portion 313 can vary in a radial direction from a center of the body 310 to an outer perimeter 334 of the body 310 (e.g., regions of the second portion 313 closer to the first portion 312 having a higher density than regions of the second portion 313 closer to the outer perimeter 334). In certain embodiments, the density and/or the fraction of open regions of the body 310 can differ from an inner first portion 312 to an outer second portion 313 (e.g., to improve osseointegration and/or vibration conduction efficiency). Certain such embodiments can comprise one or more middle portions between the inner first portion 312 and the outer second portion 313. The one or more middle portions can have corresponding densities such that the outer second portion 313 is more dense than is the middle portions and the first inner portion 312 and/or corresponding fractions of open regions such that the outer second portion 313 has a lower fraction of open regions than do the middle portions and the inner first portion 312.

In certain embodiments, the body 310 comprises a plurality of structural elements 314 (e.g., struts; scaffolding; elongate portions) and a plurality of open regions 315 between the structural elements 314. For example, as schematically illustrated by FIGS. 2A-2C, the second portion 313 of the body 310 comprises a plurality of circular structural elements 314a, a plurality of straight structural elements 314b, and a structural element 314c extending along the outer perimeter 334 of the body 310, with a plurality of open regions 315 therebetween. Various other configurations of the structural elements 314 and open regions 315 are also compatible with certain embodiments described herein. In certain embodiments, the body 310 comprises scaffold-like structures (e.g., the structural elements 314 and the open regions 315) that are configured to facilitate osseointegration of the body 310 with the recipient's bone 136.

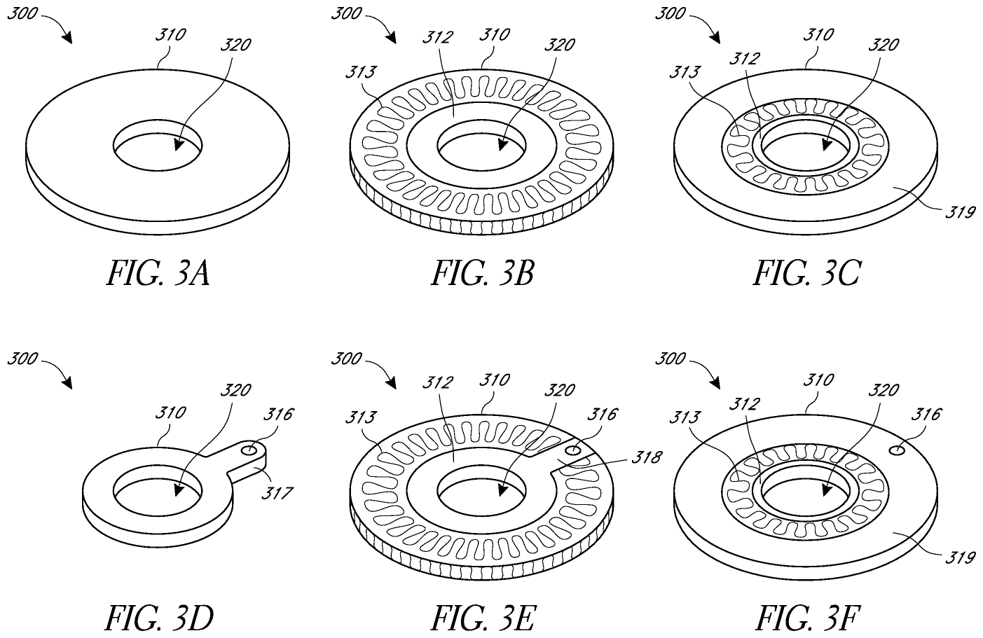

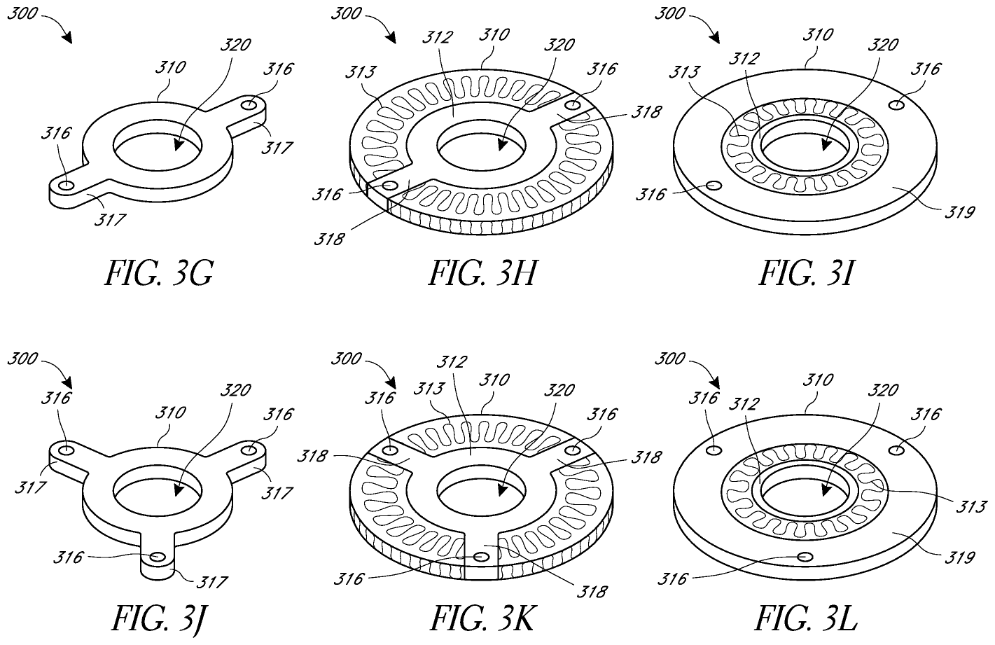

FIGS. 3A-3L schematically illustrate various example apparatus 300 in accordance with certain embodiments described herein. Each of the example apparatus 300 of FIGS. 3A-3C includes no holes configured to receive bone screws, each of the example apparatus 300 of FIGS. 3D-3F includes one hole 316 configured to receive a bone screw, each of the example apparatus 300 of FIGS. 3G-3I includes two holes 316 configured to receive two bone screws, and each of the example apparatus 300 of FIGS. 3J-3L includes three holes 316 configured to receive three bone screws. Other numbers of holes 316 for bone screws are also compatible with certain embodiments described herein. The one or more bone screws are configured to affix the body 310 to the bone 136 during osseointegration of the body 310 with the bone 136 (e.g., and subsequent to osseointegration to provide a stronger adherence of the body 310 to the bone 136).

Each of the example apparatus 300 of FIGS. 3D, 3G, and 3J includes one or more extensions 317 (e.g., arms) (e.g., extending in a radial direction away from a center of the body 310), each extension 317 having one hole 316 for a bone screw and configured to be pressed against the bone 136 by the bone screw. In certain embodiments, at least one extension 317 is configured to be compressed along its length (e.g., in the radial direction) when the apparatus 300 is inserted into the recess 500. Each of the example apparatus 300 of FIGS. 3E, 3H, and 3K includes one or more regions 318 within the second portion 313, each of which has one hole 316 for a bone screw. Each of the example apparatus 300 of FIGS. 3C, 3F, 3I, and 3L includes an outer region 319 surrounding the second portion 313. For the example apparatus 300 of FIGS. 3F, 3I, and 3L, the outer region 319 contains the one or more holes 316 for the one or more bone screws.

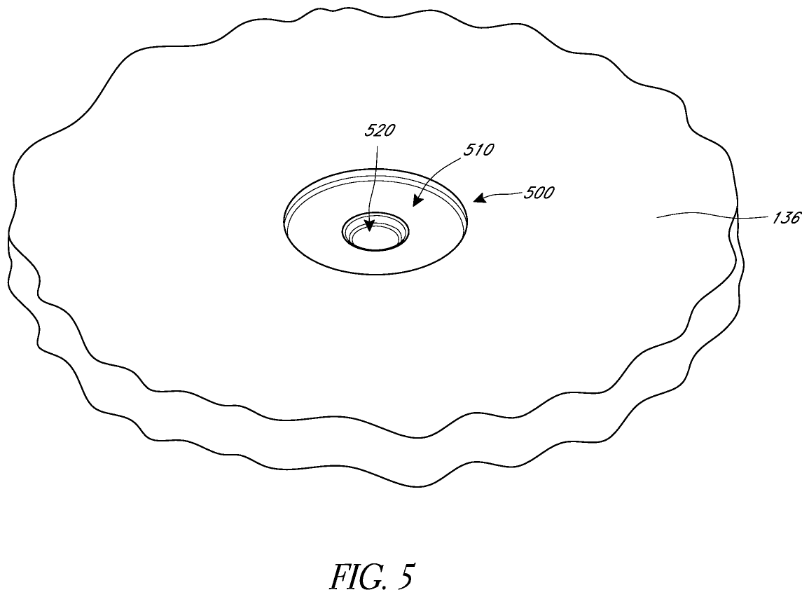

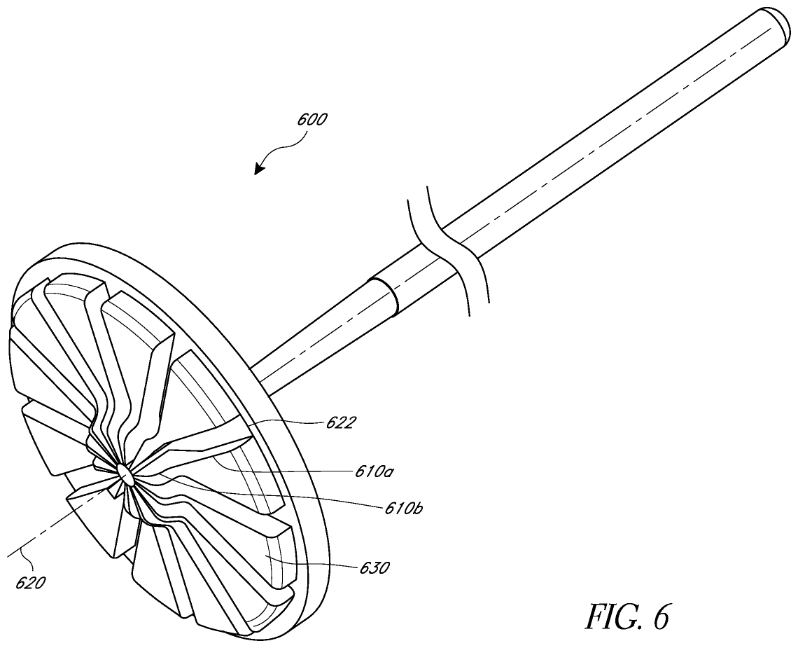

FIGS. 4A-4C schematically illustrate an example implanted apparatus 300 and an example subcutaneous acoustic transducer device 400 in accordance with certain embodiments described herein. FIG. 4A schematically illustrates a side cross-sectional view of the example apparatus 300 implanted within a recess 500 within a cortical portion of the bone 136. For example, the body 310 of the apparatus 300 can be configured to be implanted in contact with a first cortical bone surface that is recessed relative to a surrounding second cortical bone surface. FIG. 4B schematically illustrates a perspective cross-sectional view of the example apparatus 300 and example subcutaneous acoustic transducer device 400 of FIG. 4A. In both FIGS. 4A and 4B, the internal mechanisms of the acoustic transducer device 400 are not shown in detail, but are represented by a hatched region (with hatching that is different from the hatching which denotes the body 310 of the apparatus 300). FIG. 4C schematically illustrates a top view of the example subcutaneous acoustic transducer device 400 of FIG. 4A over the example apparatus 300 of FIG. 4A. FIG. 5 schematically illustrates an example recess 500 within the bone 136 in accordance with certain embodiments described herein.

In certain embodiments, the acoustic transducer device 400 comprises at least one protrusion 410 configured to be received by (e.g., to mate with; to extend at least partially within) the at least one hole 320 of the apparatus 300. For example, as schematically illustrated by FIGS. 4A and 4B, the acoustic transducer device 400 comprises a protrusion 410 (e.g., ball-like; hemispherical; concave portion) having a circular outer surface configured to be mechanically coupled to (e.g., fits at least partially within; mates with) a circular inner surface of the hole 320 with an annular contact area between the inner surface (e.g., metal surface) of the hole 320 and the outer surface (e.g., metal surface) of the protrusion 410. For another example, the hole 320 and the protrusion 410 can each have a non-circular shape such that the protrusion 410 is configured to be mechanically coupled to (e.g., fits at least partially within; mates with) the hole 320 so as to maintain a predetermined orientation of the acoustic transducer device 400 with the body 310.

As schematically illustrated by FIGS. 4A, 4B, and 5 the recess 500 comprises a first portion 510 having a first depth and a second portion 520 having a second depth, the first portion 510 surrounding the second portion 520. For example, the first portion 510 can comprise a first planar surface 512 (e.g., a cortical surface) having a first width W₁ (e.g., a first circular planar surface with an outer diameter in a range of 10 millimeters to 30 millimeters) and that is recessed relative to a surrounding region of the bone 136. The second portion 520 can comprise a second surface 522 (e.g., comprising a circular planar surface having a second width or outer diameter W₂ in a range of 3 millimeters to 20 millimeters), surrounded by the first planar surface 512. In certain embodiments, an inner diameter of the hole 320 of the body 310 is substantially equal to or greater than the outer diameter W₂ of the second surface 522, while in certain other embodiments, an inner diameter of the hole 320 of the body 310 is substantially equal to or less than the outer diameter W₂ of the second surface 522. In certain embodiments, the first portion 510 is recessed relative to the outer cortical surface 138 by a first depth D₁ in a range of 0.1 millimeter to 4 millimeters (e.g., in a range of 0.5 millimeter to 1.5 millimeter) and the second portion 520 is recessed relative to the first planar surface 512 by a second depth D₂ in a range of 0.3 millimeter to 2 millimeters (e.g., in a range of 0.5 millimeter to 1 millimeter).

In certain embodiments, there is no recess and the apparatus 300 is affixed to an outer cortical surface 138 of the bone 136. In certain such embodiments, the apparatus 300 comprises one or more holes 316 configured to receive one or more bone screws configured to affix the apparatus 300 to the outer cortical surface 138 of the bone 136 (e.g., as schematically illustrated by FIGS. 3D-3L). For example, the extensions 317 (e.g., arms) of FIGS. 3D, 3G, and 3J can be configured to bend to follow a contour of the outer cortical surface 138. For another example, the apparatus 300 can have a non-planar (e.g., curved) second surface 332 that is configured to follow a contour of the outer cortical surface 138 (e.g., by forming the apparatus 300 using additive manufacturing or three-dimensional printing using computer tomography data indicative of the shape of the recipient's outer cortical surface 138).

In certain embodiments, the recess 500 does not comprise a second portion 520 (e.g., the recess 500 has a uniform depth across the whole recess 500). In certain embodiments, at least a portion of the apparatus 300 is configured to protrude or extend above the outer cortical surface 138 of the bone 136 (e.g., the apparatus 300 is not wholly within the recess 500). For example, as schematically illustrated by FIGS. 4A and 4B, the first portion 312 of the body 310 protrudes or extends above the first surface 330 of the surrounding second portion 313 (e.g., by a distance in a range of 0.1 millimeter to 1 millimeter; by 0.5 millimeter), with the first surface 330 is substantially flush with the outer cortical surface 138 of the bone 136 and the second surface 332 is in contact with a bottom surface of the recess 500. In certain other embodiments, the apparatus 300 is configured to not protrude or extend above the outer cortical surface 138 of the bone 136 (e.g., the apparatus 300 is wholly within the recess 500; the body 310 has a thickness that is less than the first depth of the first portion 510 of the recess 500).

In certain embodiments, the recess 500 is wholly within a cortical region of the bone 136 (e.g., does not extend beyond 2 millimeters below the outer cortical surface 138 of the bone 136), while in certain other embodiments, the recess 500 extends through the cortical region of the bone 136 (e.g., extends beyond 2 millimeters below the outer cortical surface 138 of the bone 136). In certain such embodiments, the second surface 332 of the apparatus 300 is in contact with a softer, non-cortical portion of the bone 136, while the outer perimeter 334 of the body 310 is in contact with a cortical portion of the bone 136.

In certain embodiments, the hole 320 and the protrusion 410 are configured to not form a volume wholly enclosed by the inner surface of the hole 320 and the outer surface of the protrusion 410 (e.g., an enclosed zone between the apparatus 300 and the acoustic transducer device 400) when the apparatus 300 and the acoustic transducer device 400 are mechanically coupled to one another. For example, as schematically illustrated by FIGS. 4A and 4B, the outer surface of the protrusion 410 can be configured to lie on an edge of the inner surface of the hole 320 and a volume 530 below the protrusion 410 is bounded by the inner surface of the hole 320, the outer surface of the protrusion 410, and by an inner surface (e.g., the second surface 522) of the recess 500. The inner surface of the recess 500 provides a path through which body fluids can reach the volume 530 to counteract infection within the volume 530 (e.g., the bottom of the volume 530 is open to the recipient's bone 136). By avoiding forming an enclosed volume, certain such embodiments advantageously avoid enclosed zones between the apparatus 300 and the acoustic transducer device 400, thereby reducing the possibility of infection risk.

In certain other embodiments, the hole 320 and the protrusion 410 are configured to form a hermetic seal when the body 310 and the acoustic transducer device 400 are mechanically coupled to one another. For example, the second surface 332 of the body 310 can extend fully across the inner surface of the recess 500 (e.g., the hole 320 can extend only partly through the thickness of the body 310) and the hermetic seal can be between a first volume enclosed by the inner surface of the hole 320 and the outer surface of the protrusion 410 and a second volume outside the first volume. In certain embodiments, the inner surface of the hole 320 and the outer surface of the protrusion 410 are configured to mate with one another (e.g., by snap fit connection; by screw fit connection), thereby forming a hermetic seal that wholly surrounds and seals off the first volume from the second volume. By forming such a hermetic seal between the first volume and the inner surface of the recess 500 (e.g., the second surface 22), certain such embodiments advantageously ensure that there is no ingress of body fluids into the first volume, thereby reducing the possibility of infection risk.

In certain embodiments, the protrusion 410 comprises one or more curved (e.g., rounded) portions that are in mechanical communication (e.g., in contact) with corresponding one or more portions of the body 310 surrounding the hole 320. For example, the one or more curved portions of the protrusion 410 and the corresponding one or more portions of the body 310 can be configured to allow for movement of the acoustic transducer device 400 relative to the apparatus 300 (e.g., during operation of the acoustic transducer device 400) without having fixation and/or stability issues.

FIG. 6 schematically illustrates an example drilling apparatus 600 configured to be used during implantation of the example osseointegrating apparatus 300 in accordance with certain embodiments described herein. The example apparatus 600 of certain embodiments described herein advantageously facilitates simple, easy, and fast generation of the recess 500 using a single drill step.

The apparatus 600 of certain embodiments comprises a plurality of cutting edges 610 configured to be rotated about an axis 620 to machine a bone 136 of a recipient. The plurality of cutting edges 610 comprises at least a first set of the cutting edges 610a configured to machine a first planar surface 512 (e.g., a cortical surface of a first portion 510 of the recess 500) on the bone 136, the first planar surface 512 recessed relative to a surrounding region of the bone 136 (e.g., a surrounding outer cortical surface 138). By rotating the apparatus 600 about the axis 620 at sufficient speed for machining bone while pressing the cutting edges 610 against the recipient's bone 136 (e.g., in a direction along the axis 620 and perpendicular to the outer cortical surface 138 of the bone 136), the cutting edges 610 remove bone material thereby forming the recess 500. In this way, certain embodiments advantageously form the recess 500 in a single drill step.

In certain embodiments, the first planar surface 512 is recessed relative to the surrounding region of the bone 136 by a first depth in a range of 0.1 millimeter to 4 millimeters. In certain embodiments, the first set of the cutting edges 610a extend from a plane 622 perpendicular to the axis 620 by a distance substantially equal to the first depth. In certain such embodiments, when using the apparatus 600 to machine the bone 136 as described herein, the first set of the cutting edges 610a form the first portion 510 of the recess 500. In certain embodiments, the first planar surface 512 is circular with an outer diameter W₁ in a range of 10 millimeters to 30 millimeters.

In certain embodiments, the plurality of cutting edges 610 further comprises a second set of the cutting edges 610b configured to machine a second surface 522 (e.g., comprising a circular planar surface having a second width or outer diameter W₂ in a range of 3 millimeters to 20 millimeters) on the bone 136. The second surface is surrounded by the first planar surface 512 and is recessed relative to the first planar surface 512. In certain embodiments, the second set of the cutting edges 610b extend from the plane 622 perpendicular to the axis 620 by a distance substantially equal to the second depth. In certain such embodiments, when using the apparatus 600 to machine the bone 136 as described herein, the second set of the cutting edges 610b form the second portion 520 of the recess 500. In certain embodiments, the second surface 522 is recessed relative to the first planar surface 512 by a second depth in a range of 0.5 millimeter to 2 millimeters.

In certain embodiments, the first set of the cutting edges 610a and the second set of the cutting edges 610b are portions of cutting elements 630 that extend radially relative to the axis 620. As schematically illustrated by FIG. 6, each cutting element 630 has a first edge portion which is one of the cutting edges 610a and a second edge portion which is one of the cutting edges 610b.

While FIG. 6 schematically illustrates an example apparatus 600 comprising the first set of cutting edges 610a and the second set of cutting edges 610b that are configured to form a recess 500 having a first portion 510 and a second portion 520. For example, such an apparatus 600 and recess 500 can be used for an example apparatus 300 that is not configured (e.g., does not have sufficient thickness) to prevent the protrusion 410 of the acoustic transducer device 400 from contacting a surface of the recess 500 and that utilizes the further recessed second portion 520 to prevent such contact.

In certain other embodiments, the apparatus 600 comprises only the first set of cutting edges 610a and is configured to form a recess 500 having only the first portion 510 (e.g., not having a second portion 520 further recessed from the first portion 510). For example, such an apparatus 600 and recess 500 can be used for an example apparatus 300 that is configured to protrude or extend above the outer cortical surface 138 and to have sufficient thickness to prevent the protrusion 410 of the acoustic transducer device 400 from contacting a surface of the recess 500.

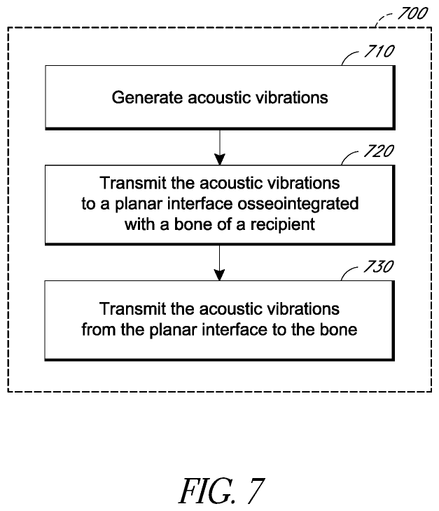

FIG. 7 is a flow diagram of an example method 700 in accordance with certain embodiments described herein. In an operational block 710, the method 700 comprises generating acoustic vibrations (e.g., by at least one microphone of an auditory prosthesis, such as a bone conduction device 400) in response to ambient sound from an environment of a recipient. In an operational block 720, the method 700 further comprises transmitting the acoustic vibrations to a planar interface (e.g., apparatus 300) in mechanical communication with (e.g., osseointegrated with) a bone 136 of a recipient. The planar interface comprises a surface receiving the acoustic vibrations (e.g., an annular surface comprising a portion of an inner surface of at least one hole 320 through the planar interface). In certain embodiments, the planar interface is at least partially recessed relative to a surrounding region of the bone 136 (e.g., an outer cortical surface 138).

In an operational block 730, the method 700 further comprises transmitting the acoustic vibrations from the planar interface to the bone 136 of the recipient. In certain embodiments, the method 700 further comprises transmitting the acoustic vibrations from the bone 136 of the recipient to the auditory sensing system of the recipient (e.g., as part of the operation of the bone conduction device 400). In certain embodiments, transmitting the acoustic vibrations from the planar interface to the bone 136 is performed prior to the planar interface being osseointegrated with the bone 136 (e.g., while one or more bone screws affix the planar interface to the bone 136). In certain embodiments, transmitting the acoustic vibrations from the planar interface to the bone 136 is performed subsequent to the planar interface being osseointegrated with the bone 136 (e.g., while one or more bone screws provide further stability to the planar interface on the bone 136).

It is to be appreciated that the embodiments disclosed herein are not mutually exclusive and may be combined with one another in various arrangements.

The invention described and claimed herein is not to be limited in scope by the specific example embodiments herein disclosed, since these embodiments are intended as illustrations, and not limitations, of several aspects of the invention. Any equivalent embodiments are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example embodiments disclosed herein, but should be defined only in accordance with the claims and their equivalents.

1. An apparatus comprising:
a planar body comprising an osseointegrating material and at least one hole configured to receive at least one protrusion of a subcutaneous acoustic transducer device, the body configured to be implanted in contact with a portion of a bone of a recipient.

2. The apparatus of claim 1, wherein the body is configured to be between the acoustic transducer device and the portion of the bone.

3. The apparatus of claim 2, wherein the acoustic transducer device comprises a vibrating actuator, the body configured to transmit acoustic vibrations from the vibrating actuator, through the at least one protrusion, to the portion of the bone.

4. The apparatus of claim 2, wherein the acoustic transducer device comprises a microphone, the body configured to provide sufficient vibration transfer between the microphone and the bone to facilitate noise cancellation to the microphone.

5. The apparatus of claim 1, wherein the body is circular and has an outer diameter in a range of 10 millimeters to 30 millimeters.

6. The apparatus of claim 1, wherein the osseointegrating material comprises titanium.

7. The apparatus of claim 1, wherein the at least one hole and the at least one protrusion are configured to not form a volume wholly enclosed by an inner surface of the at least one hole and an outer surface of the at least one protrusion when the body and the acoustic transducer device are mechanically coupled to one another.

8. The apparatus of claim 1, wherein the at least one hole and the at least one protrusion are configured to form a hermetic seal when the body and the acoustic transducer device are mechanically coupled to one another, the hermetic seal between a first volume enclosed by an inner surface of the at least one hole and an outer surface of the at least one protrusion and a second volume outside the first volume.

9. The apparatus of claim 1, wherein the at least one hole comprises a hole extending from a first surface of the body to a second surface of the body, the first surface facing towards the acoustic transducer device and the second surface facing towards and configured to contact the portion of the bone.

10. The apparatus of claim 9, wherein the body has a thickness between the first surface and the second surface, the thickness in a range of 1 millimeter to 3 millimeters.

11. The apparatus of claim 9, wherein the hole has a circular inner surface with an inner diameter in a range of 3 millimeters to 20 millimeters, the at least one protrusion of the acoustic transducer device comprises a protrusion having a circular outer surface configured to be mechanically coupled to the inner surface of the hole with an annular contact area.

12. The apparatus of claim 9, wherein the hole is non-circular and is configured to mate with the at least one protrusion so as to maintain a predetermined orientation of the acoustic transducer device with the body.

13. The apparatus of claim 1, wherein the portion of the bone comprises a first cortical bone surface that is recessed relative to a surrounding second cortical bone surface.

14. The apparatus of claim 13, wherein the body comprises a first portion surrounding the at least one hole and a second portion surrounding the first portion, the first portion having a first density and the second portion having a second density less than the first density, the first density and the second density configured to facilitate transfer of acoustic vibrations from the at least one protrusion to the bone.

15. The apparatus of claim 13, wherein the body comprises a first portion surrounding the at least one hole and a second portion surrounding the first portion, the first portion having a first fraction of open regions and the second portion having a second fraction of open regions greater than the first fraction, the first fraction and the second fraction configured to facilitate osseointegration of the body with the cortical bone.

16. The apparatus of claim 1, further comprising one or more holes configured to receive one or more bone screws, the one or more bone screws configured to affix the body to the bone during osseointegration of the body with the bone.

17. The apparatus of claim 1, wherein the at least one protrusion comprises one or more curved portions that are configured to be in mechanical communication with corresponding one or more portions of the body surrounding the hole.

18. A method comprising:
generating acoustic vibrations in response to ambient sound from an environment of a recipient;
transmitting the acoustic vibrations to an planar interface in mechanical communication with a bone of the recipient, the planar interface comprising a surface receiving the acoustic vibrations; and
transmitting the acoustic vibrations from the planar interface to the bone of the recipient.

19. The method of claim 18, wherein the planar interface is osseointegrated with the bone of the recipient.

20. The method of claim 18, wherein the planar interface is at least partially recessed relative to a surrounding region of the bone.

21. The method of claim 18, further comprising transmitting the acoustic vibrations from the bone of the recipient to the auditory sensing system of the recipient.

22. The method of claim 18, wherein generating acoustic vibrations is performed by at least one microphone of an auditory prosthesis.

23. The method of claim 18, wherein the surface receiving the acoustic vibrations is annular and comprises a portion of an inner surface of a hole through the planar interface.

24. An apparatus comprising:
a plurality of cutting edges configured to rotated about an axis to machine a portion of a bone of a recipient, the plurality of cutting edges comprising at least a first set of the cutting edges configured to machine a first planar surface on the bone, the first planar surface recessed relative to a surrounding region of the bone.

25. The apparatus of claim 24, wherein the first planar surface is recessed relative to the surrounding region of the bone by a first depth in a range of 0.1 millimeter to 4 millimeters.

26. The apparatus of claim 24, wherein the first planar surface is circular with an outer diameter in a range of 10 millimeters to 30 millimeters.

27. The apparatus of claim 24, wherein the plurality of cutting edges further comprises a second set of the cutting edges configured to machine a second surface on the bone, the second surface surrounded by the first planar surface and recessed relative to the first planar surface.

28. The apparatus of claim 27, wherein the second surface is recessed relative to the first planar surface by a second depth in a range of 0.5 millimeter to 2 millimeters.

29. The apparatus of claim 27, wherein the second surface is circular with an outer diameter in a range of 3 millimeters to 20 millimeters.
